(19) United States
(12) Patent Application Publication
(10) Pub. No.: US 2012/0071954 A1
(43) Pub. Date: Mar. 22, 2012

(54) SMALL ULTRAVIIOLET (UV) IRRADIATION MODULE

(76) Inventors: Fu-Jen Kao, Taipei City (TW); Thilo Dellwig, Taipei City (TW)

(21) Appl. No.: 12/954,377

(22) Filed: Nov. 24, 2010

(30) Foreign Application Priority Data
Sep. 17, 2010 (TW) 099131589

(51) Int. Cl. A61N 5/06; G21K 5/00

(52) U.S. Cl. 607/88; 250/492.1

(57) ABSTRACT

The present invention relates to a small ultraviolet (UV) irradiation module, at least including a solar panel (or a miniature power generating device) utilizing the photo-electric effect to convert light into electricity and an ultraviolet light-emitting device receiving electricity from solar panel to emit UV light. The present invention further relates to a clothing with the solar panel attached outside to absorb light and the ultraviolet light-emitting device attached inside to irradiate the skin for its efficient stimulation of vitamin D synthesis.

### BACKGROUND

1. Field of the Invention

The present invention relates to a small ultraviolet (UV) irradiation module that utilize solar panels to power a light-emitting device to emit UV light which irradiates the skin to facilitate the synthesizing of vitamin D.

2. Description of the Related Art

A significant number of medical publications and studies have shown that a lack of vitamin D can cause servere diseases, especially osteoporosis. It also has been confirmed that vitamin D has excellent healing effect on diseases related to spinal cord injury (SCI) and multiple sclerosis (MS).

Currently, the most efficient and best source of vitamin D is the synthesis system of the skin. The mechanism of producing vitamin D using sunlight is via the absorption of the UVB by the skin surface. Even though depending upon many factors like season, daytime, clouds and latitude of the location, exposure to sunlight provides in many cases sufficient UVB light for the facilitation of a healthy level of Vitamin D production. Under ideal conditions, a 10 minute midday sunbath will lead to the production of approximately 10,000 Internal Units (IU) of Vitamin D. The recommended minimum daily Vitamin D dose for an adult person is 1000 IU. In comparison, a glass of Vitamin D fortified milk provides about 100 IU, an egg 25 IU and a tablespoon of cod liver oil about 1,300 IU.

However, in high latitudes (latitude >37 degrees), due to atmospheric absorption and the large incidence angle of the sunlight, the UVB portion of the sunlight is not sufficient to produce a significant amount of vitamin D. Even though Vitamin D is fat soluble and some Vitamin D is stored in body fat during summer and is later used in sun-deficient periods, this is in many cases not sufficient, especially for at-risk groups like people above 60 years and people with darker skin tones, as both groups have significantly lower levels of Vitamin D production compared to young, fair-skinned Caucasians. Consequently, a significant proportion of the population living at high latitudes suffer from depression and other mental and physical illnesses closely linked to the lack of UVB radiation and thus the deficiency of Vitamin D. Therefore, providing residents at high latitudes and those who work long-term at night with sufficient UVB radiation to maintain physical and mental health is one of the major problems that the medical community is eager to solve.

### SUMMARY

Due to a lack of UVB radiation, a significant proportion of residents at high latitudes suffer from depression and other mental and physical illnesses. The main purpose of this invention is to present an Ultraviolet (UV) irradiation module that provides, at any time, the necessary quantity of UVB radiation a human body requires to maintain physical and mental health.

For the above-mentioned purpose, the small UV irradiation module disclosed in this invention includes at least:

A solar panel that, by means of the photoelectric effect, transforms light energy of the ambient (indoor or outdoor) light directly into electric energy;

An Ultraviolet light-emitting device lighting module attached to a patient's body in a way that enables a constant low-dosage UVB exposure of the patient's skin, which utilizes the electric energy produced by the solar panel to produce UV light, without any negative impact on the patient's comfort and mobility. In addition to the solar panel, the electric energy mentioned in this invention can also be produced by small power generators such as batteries, induction generators or thermoelectric generators.

In one embodiment, the lightning module and the solar panel can be integrated into a garment or cloth, so the patient can wear the garment for a longer period of time, e.g. a work shift in the office.

The number of lightning modules integrated in a garment can be varied upon the individual necessities of the patient.

The solar panels consist preferably of flexible solar cells, like CIGS or Polymer solar cells and can be integrated into the garment independent of the location of the lightning modules, electrically connected to the lightning modules by conventional wires or woven electronic textile.

The size and nominal power production of the solar panels can be varied according to the number of lightning modules and the intensity and spectrum of the available ambient light.

The detailed features and methods of the present invention are described thoroughly below, including the relevant figures.

### DETAILED DESCRIPTION

The present invention incorporates the use of lightning modules comprising of at least one UVB light emitting diode (LED) and one or more solar panels used as power sources for the lightning modules and electrically wired to the lightning modules.

FIG. 1 shows the schematic diagram of the hardware of this invention.

The small UV irradiation module (1) of the present invention includes at least:

A solar panel (11) with a positive terminal (111) and a negative terminal (112) that transforms light energy (2) directly into electric energy by means of the photoelectric effect. The solar panel (11) is a pliable solar panel such as a CIGS solar panel, an amorphous silicon solar panel or a CdTe cadmium telluride solar panel.

An Ultraviolet light-emitting device (12) that has its anode and cathode connected in tandem with the positive terminal (111) and negative terminal (112) of the solar panel (11) in order to receive the electric energy sent by the solar panel (11) and produce UV light. The UV light-emitting device (12) is a UVB LED with a wavelength between 280 nm and 320 nm.

Solar panels (11) produce electrical energy from incoming light. Limited by the band gap of the solar panel's semiconducting material, typically 1.1 eV, the wavelength of the incoming light has to be below 1200 nm, which is given for all of the visible and ultra-violet spectrum and a good part of the infrared spectrum. Typical UVB LED's require 110 mW of electrical power. This power can be produced under sunlight by a CIGS solar cell with an efficiency of 10% and an area of 10 cm² (3.3 cm×3.3 cm). For non-window room indoor illuminations (300 W conventional light bulb or equivalent), roughly 0.5 m² (71 cm×71 cm) are required. For typical rooms having windows, a required solar panel area of 100 cm² (10 cm×10 cm) can be expected.

In addition to the solar panel (11), the electric energy mentioned in this invention can also be produced by small power generators such as batteries, induction generators or thermoelectric generators.

FIG. 2 shows the schematic diagram of the preferred embodiment of the present invention.

In this embodiment, the small UV irradiation module (1) is combined with figure-clinging clothes (3). The solar panel (11) of the small UV irradiation module (1) is set outside of the figure-clinging clothes (3) to absorb solar energy (2) and the UV light-emitting device (12) of the small UV irradiation module (1) is set inside the figure-clinging clothes (3) near the skin in order to stimulate the synthesis of Vitamin D in the skin.

One LED manufacturer supplying said LED's is Sensor Electronic Technology Inc. (USA). The solar panels preferably consist of flexible solar cells. To date, the flexible solar cells with the highest efficiency are CIGS based thin-film solar cells with efficiency above 10%. One CIGS based solar cell manufacturer is Global Solar Energy (USA). The lightning modules and solar panels are preferably integrated into a garment and connected through woven electronic textile. One woven electronic textile producing company is Gorix Ltd. (UK).

While this invention has been particularly shown and described with references to preferred embodiments thereof, it will be understood by those skilled in the art that various changes in form and details may be made therein without departing from the scope of the invention encompassed by the appended claims.

### BRIEF DESCRIPTION OF THE DRAWINGS

FIG. 1 is the schematic diagram of the hardware of this invention.

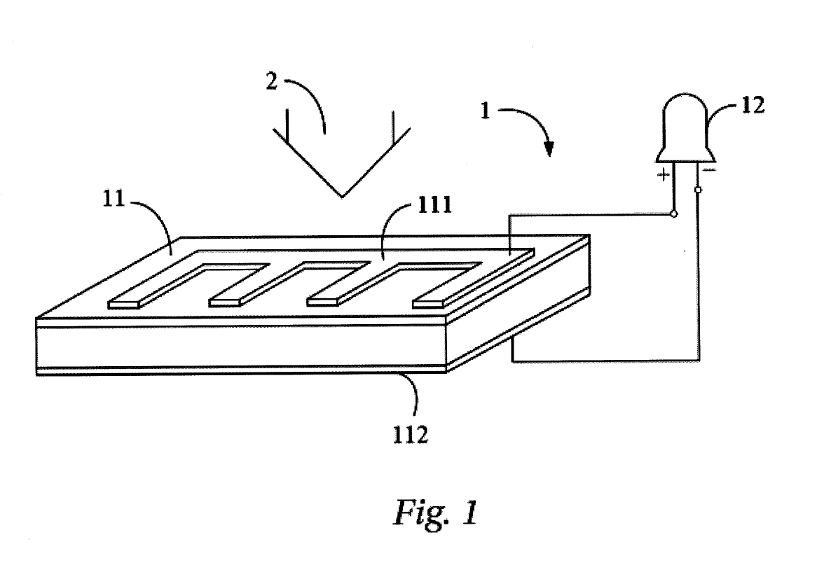

FIG. 2 is the schematic diagram of the preferred embodiment of this invention.

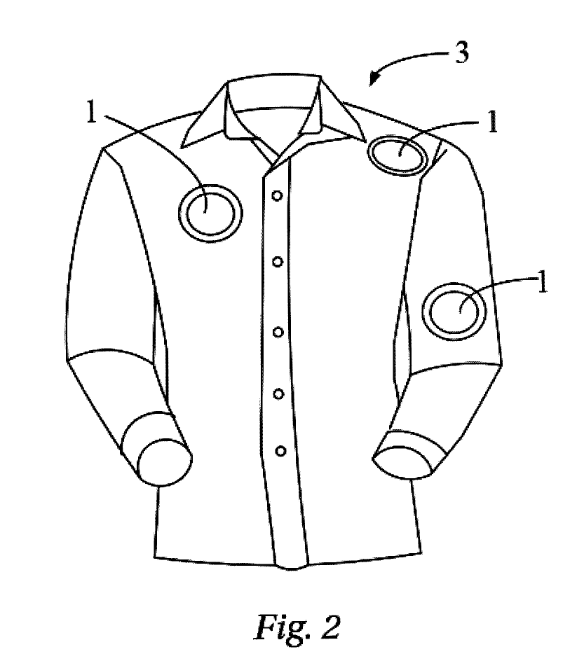

### DESCRIPTION OF MAIN COMPONENTS

| | |
|---|---|
| 1 small UV irradiation module | |
| 11 solar panel | |
| 111 positive terminal | 112 negative terminal |
| 12 UV light-emitting device | |
| 2 solar energy | |
| 3 figure-clinging clothes | |

What is claimed is:

1. A small UV irradiation module, which comprises:
a solar panel that, by means of the photoelectric effect, transforms light energy directly into electric energy; and
an Ultraviolet light-emitting device that receives the electric energy from the solar panel to produce UV light.

2. The small UV irradiation module as in claim 1, wherein the solar panel is a pliable solar panel.

3. The small UV irradiation module as in claim 2, wherein the solar panel can be a CIGS solar panel, an amorphous silicon solar panel or a CdTe cadmium telluride solar panel.

4. The small UV irradiation module as in claim 1, wherein the UV light-emitting device is a UVB LED.

5. The small UV irradiation module as in claim 4, wherein the wavelength of the UV light-emitting device is between 280 nm and 320 nm.

6. A small UV irradiation module, which comprises:
a solar panel that, by means of the photoelectric effect, transforms light energy directly into electric energy;
an Ultraviolet light-emitting device that receives the electric energy from the solar panel to produce UV; and
a figure-clinging cloth, to which the solar panel is set outside to absorb solar energy and the UV light-emitting device of the small UV irradiation module is set inside near the skin to stimulate the synthesis of Vitamin D in the skin.

7. The small UV irradiation module as in claim 6, wherein the solar panel is a pliable solar panel.

8. The small UV irradiation module as in claim 7, wherein the solar panel can be a CIGS solar panel, an amorphous silicon solar panel or a CdTe cadmium telluride solar panel.

9. The small UV irradiation module as in claim 6, wherein the UV light-emitting device is a UVB LED.

10. The small UV irradiation module as in claim 9, wherein the wavelength of the UV light-emitting device is between 280 nm and 320 nm.

11. A small UV irradiation module, which comprises:
a small power generator to generate electricity; and
an UV light-emitting device to receive the electric power generated by the small power generator to produce UV.

12. The small UV irradiation module as in claim 11, wherein the small power generator is selected from the group consisting of a battery, an induction generator, and a thermoelectric generator.
